(19) United States
(12) Patent Application Publication
(10) Pub. No.: US 2010/0029681 A1
(43) Pub. Date: Feb. 4, 2010

(54) HETEROCYCLIC COMPOUNDS AS CALCIUM CHANNEL BLOCKERS

(76) Inventors: Hassan Pajouhesh, West Vancouver (CA); Yanbing Ding, Richmond (CA)

Correspondence Address: MORRISON & FOERSTER LLP, 12531 HIGH BLUFF DRIVE, SUITE 100, SAN DIEGO, CA, 92130-2040, US

(21) Appl. No.: 12/301,673

(22) PCT Filed: May 25, 2007
(86) PCT No.: PCT/CA2007/000943

§ 371 Date: May 7, 2009

Related U.S. Application Data
(60) Provisional application No. 60/808,717, filed on May 26, 2006.

(51) Int. Cl. A61K 31/496; C07D 417/02; A61P 29/00

(52) U.S. Cl. 514/254.02; 544/368; 544/369

(57) ABSTRACT

Methods and compounds effective in ameliorating conditions characterized by unwanted calcium channel activity, particularly unwanted N-type calcium channel activity are disclosed. Specifically, a series of heterocyclic compounds are disclosed of the general formula (1) where X¹ and X² are linkers and W is an optionally substituted imidazolyl, oxazolyl, thiazolyl, benzimidazolyl, benzoxazolyl, or benzothiazolyl.

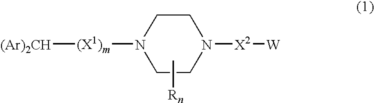

### TECHNICAL FIELD

The invention relates to compounds useful in treating conditions associated with calcium channel function, and particularly conditions associated with N-type calcium channel activity. More specifically, the invention concerns compounds containing piperazine derivatives that are useful in treatment of conditions such as stroke and pain.

### BACKGROUND ART

The entry of calcium into cells through voltage-gated calcium channels mediates a wide variety of cellular and physiological responses, including excitation-contraction coupling, hormone secretion and gene expression (Miller, R. J., Science (1987) 235:46-52; Augustine, G. J. et al., Annu Rev Neurosci (1987) 10: 633-693). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter and calcium channels, which also affects neurite outgrowth and growth cone migration in developing neurons.

Calcium channels have been shown to mediate the development and maintenance of the neuronal sensitization processes associated with neuropathic pain, and provide attractive targets for the development of analgesic drugs (reviewed in Vanegas, H. & Schaible, H-G., Pain (2000) 85: 9-18). All of the high-threshold Ca channel types are expressed in the spinal cord, and the contributions of L-, N and P/Q-types in acute nociception are currently being investigated. In contrast, examination of the functional roles of these channels in more chronic pain conditions strongly indicates a pathophysiological role for the N-type channel (reviewed in Vanegas & Schaible (2000) supra).

Two examples of either FDA-approved or investigational drugs that act on N-type channel are gabapentin and ziconotide. Gabapentin, 1-(aminomethyl)cyclohexaneacetic acid (Neurontin®), is an anticonvulsant originally found to be active in a number of animal seizure models (Taylor, C. P. et al., Epilepsy Res (1998) 29: 233-249). Subsequent work has demonstrated that gabapentin is also successful at preventing hyperalgesia in a number of different animal pain models, including chronic constriction injury (CCl), heat hyperalgesia, inflammation, diabetic neuropathy, static and dynamic mechanoallodynia associated with postoperative pain (Taylor, et al. (1998); Cesena, R. M. & Calcutt, N. A., Neurosci Lett (1999) 262: 101-104; Field, M. J. et al., Pain (1999) 80: 391-398; Cheng, J-K., et al., Anesthesiology (2000) 92: 1126-1131; Nicholson, B., Acta Neurol Scand (2000) 101: 359-371).

While its mechanism of action is not completely understood, current evidence suggests that gabapentin does not directly interact with GABA receptors in many neuronal systems, but rather modulates the activity of high threshold calcium channels. Gabapentin has been shown to bind to the calcium channel α₂δ ancillary subunit, although it remains to be determined whether this interaction accounts for its therapeutic effects in neuropathic pain.

In humans, gabapentin exhibits clinically effective anti-hyperalgesic activity against a wide ranging of neuropathic pain conditions. Numerous open label case studies and three large double blind trials suggest gabapentin might be useful in the treatment of pain. Doses ranging from 300-2400 mg/day were studied in treating diabetic neuropathy (Backonja, M. et al., JAMA (1998) 280:1831-1836), postherpetic neuralgia (Rowbotham, M. et al., JAMA (1998) 280: 1837-1842), trigeminal neuralgia, migraine and pain associated with cancer and multiple sclerosis (Di Trapini, G. et al., Clin Ter (2000) 151: 145-148; Caraceni, A. et al., J Pain & Symp Manag (1999) 17: 441-445; Houtchens, M. K. et al., Multiple Sclerosis (1997) 3: 250-253; see also Magnus, L., Epilepsia (1999) 40(Suppl 6): S66-S72; Laird, M. A. & Gidal, B. E., Annal Pharmacotherap (2000) 34: 802-807; Nicholson, B., Acta Neurol Scand (2000) 101: 359-371).

Ziconotide (Prialt®; SNX-111) is a synthetic analgesic derived from the cone snail peptide Conus magus MVIIA that has been shown to reversibly block N-type calcium channels. In a variety of animal models, the selective block of N-type channels via intrathecal administration of Ziconotide significantly depresses the formalin phase 2 response, thermal hyperalgesia, mechanical allodynia and post-surgical pain (Malmberg, A. B. & Yaksh, T. L., J Neurosci (1994) 14: 4882-4890; Bowersox, S. S. et al., J Pharmacol Exp Ther (1996) 279: 1243-1249; Sluka, K. A., J Pharmacol Exp Ther (1998) 287:232-237; Wang, Y-X. et al., Soc Neurosci Abstr (1998) 24: 1626).

Ziconotide has been evaluated in a number of clinical trials via intrathecal administration for the treatment of a variety of conditions including post-herpetic neuralgia, phantom limb syndrome, HIV-related neuropathic pain and intractable cancer pain (reviewed in Mathur, V. S., Seminars in Anesthesia, Perioperative medicine and Pain (2000) 19: 67-75). In phase II and III clinical trials with patients unresponsive to intrathecal opiates, Ziconotide has significantly reduced pain scores and in a number of specific instances resulted in relief after many years of continuous pain. Ziconotide is also being examined for the management of severe post-operative pain as well as for brain damage following stroke and severe head trauma (Heading, C., Curr Opin CPNS Investigational Drugs (1999) 1: 153-166). In two case studies Ziconotide has been further examined for usefulness in the management of intractable spasticity following spinal cord injury in patients unresponsive to baclofen and morphine (Ridgeway, B. et al., Pain (2000) 85: 287-289). In one instance Ziconotide decreased the spasticity from the severe range to the mild to none range with few side effects. In another patient Ziconotide also reduced spasticity to the mild range although at the required dosage significant side effects including memory loss, confusion and sedation prevented continuation of the therapy.

U.S. Pat. Nos. 6,011,035; 6,294,533; 6,310,059; and 6,492,375; PCT publications WO 01375 and WO 01/45709; PCT publications based on PCT CA 99/00612, PCT CA 00/01586; PCT CA 00/01558; PCT CA 00/01557; PCT CA 2004/000535; and PCT CA 2004/000539, and U.S. patent application Ser. Nos. 10/746,932 filed 23 Dec. 2003; 10/746,933 filed 23 Dec. 2003; 10/409,793 filed 8 Apr. 2003; 10/409,868 filed 8 Apr. 2003; 10/655,393 filed 3 Sep. 2003; 10/821,584 filed 9 Apr. 2004; and 10/821,389 filed 9 Apr. 2004 disclose calcium channel blockers where a piperidine or piperazine ring is substituted by various aromatic moieties.

U.S. Pat. No. 5,646,149 describes calcium channel antagonists of the formula A-Y-B wherein B contains a piperazine or piperidine ring directly linked to Y. An essential component of these molecules is represented by A, which must be an antioxidant; the piperazine or piperidine itself is said to be important. The exemplified compounds contain a benzhydryl substituent, based on known calcium channel blockers (see below). U.S. Pat. No. 5,703,071 discloses compounds said to be useful in treating ischemic diseases. A mandatory portion of the molecule is a tropolone residue, with substituents such as piperazine derivatives, including their benzhydryl derivatives. U.S. Pat. No. 5,428,038 discloses compounds indicated to exhibit a neural protective and antiallergic effect. These compounds are coumarin derivatives which may include derivatives of piperazine and other six-membered heterocycles. A permitted substituent on the heterocycle is diphenylhydroxymethyl. U.S. Pat. No. 6,458,781 describes 79 amides as calcium channel antagonists though only a couple of which contain both piperazine rings and benzhydryl moieties. Thus, approaches in the art for various indications which may involve calcium channel blocking activity have employed compounds which incidentally contain piperidine or piperazine moieties substituted with benzhydryl but mandate additional substituents to maintain functionality.

Certain compounds containing both benzhydryl moieties and piperidine or piperazine are known to be calcium channel antagonists and neuroleptic drugs. For example, Gould, R. J., et al., Proc Natl Acad Sci USA (1983) 80:5122-5125 describes antischizophrenic neuroleptic drugs such as lidoflazine, fluspirilene, pimozide, clopimozide, and penfluridol. It has also been shown that fluspirilene binds to sites on L-type calcium channels (King, V. K., et al., J Biol Chem (1989) 264:5633-5641) as well as blocking N-type calcium current (Grantham, C. J., et al., Brit J Pharmacol (1944) 111:483-488). In addition, Lomerizine, as developed by Kanebo, K. K., is a known calcium channel blocker. However, Lomerizine is not specific for N-type channels. A review of publications concerning Lomerizine is found in Dooley, D., Current Opinion in CPNS Investigational Drugs (1999) 1:116-125.

All patents, patent applications and publications are herein incorporated by reference in their entirety.

### DISCLOSURE OF THE INVENTION

The invention relates to compounds useful in treating conditions modulated by calcium channel activity and in particular conditions mediated by N-type channel activity.

The compounds of the invention are heterocyclic compounds with substituents that enhance the calcium channel blocking activity of the compounds. Thus, in one aspect, the invention is directed to a method of treating conditions mediated by calcium channel activity by administering to patients in need of such treatment a compound of formula (1):

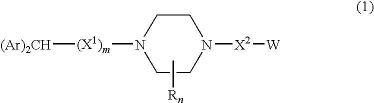

or a pharmaceutically acceptable salt or conjugate thereof, wherein:

W is an optionally substituted imidazolyl, thiazolyl, oxazolyl, benzimidazolyl, benzothiazolyl or benzoxazolyl;

X¹ is an optionally substituted alkylene (1-6C), alkenylene (2-6C), alkynylene (2-6C), heteroalkylene (2-6C), heteroalkenylene (2-6C), or heteroalkynylene (2-6C);

X² is an optionally substituted alkylene (3-6C) or heteroalkylene (2-6C);

each Ar is independently an optionally substituted aromatic or heteroaromatic ring;

each R is independently ═O, ═NOR′, halo, CN, OR′, SR′, SOR′, SO₂R′, NR′₂, NR′(CO)R′, or NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-10C); or R may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-1° C.), heteroaryl (5-12C), O-aryl (6-1° C.), O-heteroaryl (5-12C) and C₆-C₁₂-aryl-C₁-C₆-alkyl;

n is 0-4;

m is 0-1; and

wherein the optional substituents on each Ar and W are independently selected from halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-1° C.); or the optional substituent may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-1° C.), heteroaryl (5-12C), O-aryl (6-10C), O-heteroaryl (5-12C) and C6-C12-aryl-C1-C6-alkyl; and

wherein the optional substituents on each X¹ and X² are independently selected from ═O, ═NOR′, halo, CN, OR′, SR′, SOR′, SO₂R′, NR′₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-10C); or R may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12C), O-aryl (6-10C), O-heteroaryl (5-12C) and C6-C12-aryl-C1-C6-alkyl.

The invention is also directed to compounds of formula (1) useful to modulate calcium channel activity, particularly N-type channel activity, and to methods of treating such conditions with these compounds. The invention is also directed to the use of these compounds for the preparation of medicaments for the treatment of conditions requiring modulation of calcium channel activity, and in particular N-type calcium channel activity. In another aspect, the invention is directed to pharmaceutical compositions containing the compounds of formula (1) and to the use of these compositions for treating conditions requiring modulation of calcium channel activity, and particularly N-type calcium channel activity.

### DETAILED DESCRIPTION

As used herein, the term “alkyl,” “alkenyl” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. Typically, the alkyl, alkenyl and alkynyl groups contain 1-6C (alkyl) or 2-6C (alkenyl or alkynyl). In some embodiments, they contain 1-4C or 1-2C (alkyl); or 2-4C (alkenyl or alkynyl). Further, any hydrogen atom on one of these groups can be replaced with a halogen atom, and in particular a fluoro or chloro, and still be within the scope of the definition of alkyl, alkenyl and alkynyl. For example, CF₃ is a 1C alkyl. These groups may be also be substituted by other substituents.

Heteroalkyl, heteroalkenyl and heteroalkynyl are similarly defined and contain at least one carbon atom but also contain one or more O, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In preferred embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In preferred embodiments, the heteroatom is O or N. For greater certainty, to the extent that alkyl is defined as 1-6C, then the corresponding heteroalkyl contains 2-6 C, N, O, or S atoms such that the heteroalkyl contains at least one C atom and at least one heteroatom. Similarly, when alkyl is defined as 1-6C or 1-4C, the heteroform would be 2-6C or 2-4C respectively, wherein one C is replaced by O, N or S. Accordingly, when alkenyl or alkynyl is defined as 2-6C (or 2-4C), then the corresponding heteroform would also contain 2-6 C, N, O, or S atoms (or 2-4) since the heteroalkenyl or heteroalkynyl contains at least one carbon atom and at least one heteroatom. Further, heteroalkyl, heteroalkenyl or heteroalkynyl substituents may also contain one or more carbonyl groups. Examples of heteroalkyl, heteroalkenyl and heteroalkynyl substituents include CH₂OCH₃, CH₂N(CH₃)₂, CH₂OH, (CH₂)ₙNR₂, OR, COOR, CONR₂, (CH₂)ₙOR, (CH₂)ₙCOR, (CH₂)ₙCOOR, (CH₂)ₙSR, (CH₂)ₙSOR, (CH₂)ₙSO₂R, (CH₂)ₙCONR₂, NRCOR, NRCOOR, OCONR₂, OCOR and the like wherein the substituent contains at least one C and the size of the substituent is consistent with the definition of alkyl, alkenyl and alkynyl.

As used herein, the terms “alkylene,” “alkenylene” and “alkynylene” refer to divalent groups having a specified size, typically 1-4C or 1-6C for the saturated groups and 2-4C or 2-6C for the unsaturated groups. They include straight-chain, branched-chain and cyclic forms as well as combinations of these, containing only C and H when unsubstituted. Because they are divalent, they can link together two parts of a molecule, as exemplified by X¹ and X² in formula (1). Examples include methylene, ethylene, propylene, cyclopropan-1,1-diyl, ethylidene, 2-butene-1,4-diyl, and the like. These groups can be substituted by the groups typically suitable as substituents for alkyl, alkenyl and alkynyl groups as set forth herein. Thus C═O is a C1 alkylene that is substituted by ═O, for example.

Heteroalkylene, heteroalkenylene and heteroalkynylene are similarly defined as divalent groups having a specified size, typically 2-4C or 2-6C for the saturated groups and 2-4C or 2-6C for the unsaturated groups. They include straight chain, branched chain and cyclic groups as well as combinations of these, and they further contain at least one carbon atom but also contain one or more O, S or N heteroatoms or combinations thereof within the backbone residue, whereby each heteroatom in the heteroalkylene, heteroalkenylene or heteroalkynylene group replaces one carbon atom of the alkyl, alkenyl or alkynyl group to which the heteroform corresponds. As is understood in the art, these heteroforms do not contain more than three contiguous heteroatoms.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, 2-, 3- or 4-pyridyl, indolyl, 2- or 4-pyrimidyl, pyridazinyl, benzothiazolyl or benzimidazolyl. Even more particularly, such moiety is phenyl, pyridyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of 1-8C or more particularly 1-6C or 1-4C when saturated or 2-8C, 2-6C or 2-4C when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(6-12C)alkyl(1-8C), aryl(6-12C)alkenyl(2-8C), or aryl(6-12C)alkynyl(2-8C), plus the heteroforms. A typical example is phenylmethyl, commonly referred to as benzyl.

For greater certainty, in some embodiments, W is an optionally substituted member selected from the following ring systems:

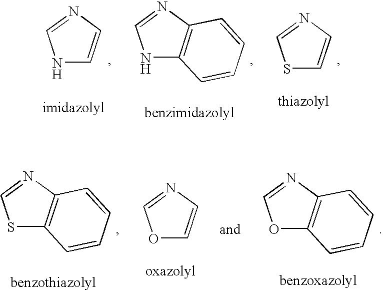

In such embodiments, X² frequently attaches to the five membered ring, and in some embodiments X² attaches to the carbon atom of the five-membered ring that is between the two heteroatoms of that ring. In other embodiments, X² may attach to a nitrogen of an imidazole or benzimidazole ring.

Typical optional substituents on aromatic or heteroaromatic groups include independently halo, CN, NO₂, CF₃, COOR′, CONR′₂, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10C); or the substituent may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12C), O-aryl (6-1° C.), O-heteroaryl (5-12C) and C6-C12-aryl-C1-C6-alkyl.

Optional substituents on a non-aromatic group, are typically selected from ═O, ═NOR′, halo, CN, OR′, SR′, SOR′, SO₂R′, NR′₂, NR′(CO)R′, or NR′SO₂R′, wherein each R′ is independently H or an optionally substituted group selected from alkyl (1-6C), heteroaryl (5-12C), and aryl (6-10C); or it may be alkyl (1-6C), alkenyl (2-6C), or alkynyl (2-6C), heteroalkyl (2-6C), heteroalkenyl (2-6C), heteroalkynyl (2-6C), aryl (6-1° C.), heteroaryl (5-12C), O-aryl (5-10C), O-heteroaryl (5-12C) and C6-C12-aryl-C1-C6-alkyl. For greater certainty, two substituents on the same nitrogen or on adjacent nitrogen or carbon atoms can form a 5-7 membered ring which may contain one or two additional heteroatoms selected from N, O and S.

Halo may be any halogen atom, especially F, Cl, Br, or I, and more particularly it is fluoro or chloro.

In general, any alkyl, alkenyl, alkynyl, or aryl (including all heteroforms defined above) group contained in a substituent may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included.

There may be from 0-4 substituents (defined as R) on the central piperazine or piperidine ring and more particularly 0-2 substituents. Each R may independently be ═O, alkyl, alkenyl, alkynyl, heteroalkyl, heteroalkenyl, heteroalkynyl, aryl, heteroaryl, alkylaryl, O-aryl, O-heteroaryl, halo, CN, OH, NO₂, or NH₂. Where it makes sense chemically, each of these groups (other than H) can be substituted. In more particular embodiments, R may be 1-6C alkyl or heteroalkyl, more particularly a 1-4C alkyl or heteroalkyl. For example, R may be CH₃, CH₂OH, CH₂OCH₃, CH₂OCH₂COOH, COOH, CH₂OCH₂CH₂OH, CH₂N(CH₃)₂, CH₂—O—(CH₂)₂N(CH₃)₂, COOCH₂CH₂N(CH₃)₂, COO(CH₂)COOH. One or more R groups may also be ═O, in which case n is typically 1 or 2.

The two Ar groups may be the same or different; in some embodiments they are the same. In certain embodiments each Ar represents phenyl, so Ar₂CH— represents a benzhydryl, and each phenyl ring may independently be substituted or unsubstituted. In certain embodiments, each Ar represents phenyl and both phenyl rings have the same substitution pattern. In certain embodiments at least one, and frequently both, phenyl rings in such embodiments have at least one halo substituent. In other embodiments, Ar₂CH represents an unsubstituted benzhydryl.

X¹ in formula (1) represents an optionally substituted alkylene (1-6C), alkenylene (2-6C), alkynylene (2-6C), heteroalkylene (2-6C), heteroalkenylene (2-6C), or heteroalkynylene (2-6C). In certain embodiments, m is 0 so X¹ is absent. In other embodiments, m is 1 and X¹ represents an alkylene (1-6C) or heteroalkylene (2-6C) that may be substituted or unsubstituted. In certain of these embodiments, X¹ is substituted with a group such as ═O, which may be adjacent to the nitrogen of the piperazine ring to which X¹ is attached.

X² is an optionally substituted alkylene (3-6C) or heteroalkylene (2-6C), and in some embodiments, X² comprises an amide linkage. One particular embodiment of X² is CH₂CONH.

In one aspect, the invention provides a compound of formula (2):

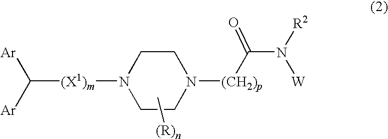

wherein Ar, X¹, m, R, n, and W are as defined for formula (1);
p is 0-4;
and R² represents H or optionally substituted alkyl (1-4C) or heteroalkyl (2-6C).

In compounds of formula (2), R² is sometimes H or methyl, and p is often 1-3. In certain embodiments of the compounds of formula (2), m is 0 and p is 1 or 2. Just as in compounds of formula (1), the two Ar groups may be the same or different, and in some embodiments each Ar represents a phenyl ring that may be unsubstituted, or may be substituted, preferably with one or more groups such as halo, methyl, trifluormethyl, or methoxy.

In some preferred embodiments, two or more of the particularly described groups are combined into one compound: it is often suitable to combine one of the specified embodiments of one feature as described above with a specified embodiment or embodiments of one or more other features as described above. For example, a specified embodiment includes W is imidazolyl, and another specified embodiment has both Ar as optionally substituted phenyl groups (i.e., Ar₂CH is an optionally substituted benzhydryl). Thus one preferred embodiment combines both of these features together, i.e., W is imidazolyl in combination with both Ar representing optionally substituted benzhydryl. In some specific embodiments, n is 0 and in others n is 1. Thus additional preferred embodiments include n=0 in combination with any of the preferred combinations set forth above; other preferred combinations include n=1 in combination with any of the preferred combinations set forth above.

The compounds of the invention may have ionizable groups so as to be capable of preparation as salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed.

Compounds of formula (1) are also useful for the manufacture of a medicament useful to treat conditions characterized by undesired N-type calcium channel activities.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to the compounds of formula (1) when modified so as to be included in a conjugate of this type.

### MODES OF CARRYING OUT THE INVENTION

The compounds of formula (1) are useful in the methods of the invention and exert their desirable effects through their ability to modulate the activity of calcium channels, particularly the activity of N-type calcium channels. This makes them useful for treatment of certain conditions where modulation of N-type calcium channels is desired, including: chronic and acute pain; mood disorders such as anxiety, depression, and addiction; neurodegenerative disorders; gastrointestinal disorders such as inflammatory bowel disease and irritable bowel syndrome; genitourinary disorders such as urinary incontinence, interstitial colitis and sexual dysfunction; neuroprotection such as cerebral ischemia, stroke and traumatic brain injury; and metabolic disorders such as diabetes and obesity.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

Anxiety as used herein includes but is not limited to the following conditions: generalized anxiety disorder, social anxiety disorder, panic disorder, obsessive-compulsive disorder, and post-traumatic stress syndrome. Addiction includes but is not limited to dependence, withdrawal and/or relapse of cocaine, opioid, alcohol and nicotine.

Neurodegenerative disorders as used herein include Parkinson's disease, Alzheimer's disease, multiple sclerosis, neuropathies, Huntington's disease and amyotrophic lateral sclerosis (ALS).

For greater certainty, in treating osteoarthritic pain, joint mobility will also improve as the underlying chronic pain is reduced. Thus, use of compounds of the present invention to treat osteoarthritic pain inherently includes use of such compounds to improve joint mobility in patients suffering from osteoarthritis.

It is known that calcium channel activity is involved in a multiplicity of disorders, and particular types of channels are associated with particular conditions. The association of N-type channels in conditions associated with neural transmission would indicate that compounds of the invention which target N-type receptors are most useful in these conditions. Many of the members of the genus of compounds of formula (1) exhibit high affinity for N-type channels. Thus, as described below, they are screened for their ability to interact with N-type channels as an initial indication of desirable function. It is particularly desirable that the compounds exhibit IC₅₀ values of <1 μM. The IC₅₀ is the concentration which inhibits 50% of the calcium, barium or other permeant divalent cation flux at a particular applied potential.

There are three distinguishable types of calcium channel inhibition. The first, designated “open channel blockage,” is conveniently demonstrated when displayed calcium channels are maintained at an artificially negative resting potential of about −100 mV (as distinguished from the typical endogenous resting maintained potential of about −70 mV). When the displayed channels are abruptly depolarized under these conditions, calcium ions are caused to flow through the channel and exhibit a peak current flow which then decays. Open channel blocking inhibitors diminish the current exhibited at the peak flow and can also accelerate the rate of current decay.

This type of inhibition is distinguished from a second type of block, referred to herein as “inactivation inhibition.” When maintained at less negative resting potentials, such as the physiologically important potential of −70 mV, a certain percentage of the channels may undergo conformational change, rendering them incapable of being activated—i.e., opened—by the abrupt depolarization. Thus, the peak current due to calcium ion flow will be diminished not because the open channel is blocked, but because some of the channels are unavailable for opening (inactivated). “Inactivation” type inhibitors increase the percentage of receptors that are in an inactivated state.

A third type of inhibition is designated “resting channel block”. Resting channel block is the inhibition of the channel that occurs in the absence of membrane depolarization, that would normally lead to opening or inactivation. For example, resting channel blockers would diminish the peak current amplitude during the very first depolarization after drug application without additional inhibition during the depolarization.

In order to be maximally useful in treatment, it is also helpful to assess the side reactions which might occur. Thus, in addition to being able to modulate a particular calcium channel, it is desirable that the compound has very low activity with respect to the HERG K⁺ channel which is expressed in the heart. Compounds that block this channel with high potency may cause reactions which are fatal. Thus, for a compound that modulates the calcium channel, it should also be shown that the HERG K⁺ channel is not inhibited. Similarly, it would be undesirable for the compound to inhibit cytochrome p450 since this enzyme is required for drug detoxification. Finally, the compound will be evaluated for calcium ion channel type specificity by comparing its activity among the various types of calcium channels, and specificity for one particular channel type is preferred. The compounds which progress through these tests successfully are then examined in animal models as actual drug candidates.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Typical assays are described hereinbelow in Examples 3 and 4.

Libraries and Screening

The compounds of the invention can be synthesized individually using methods known in the art per se, or as members of a combinatorial library.

Synthesis of combinatorial libraries is now commonplace in the art. Suitable descriptions of such syntheses are found, for example, in Wentworth, Jr., P., et al., Current Opinion in Biol. (1993) 9:109-115; Salemme, F. R., et al., Structure (1997) 5:319-324. The libraries contain compounds with various substituents and various degrees of unsaturation, as well as different chain lengths. The libraries, which contain, as few as 10, but typically several hundred members to several thousand members, may then be screened for compounds which are particularly effective against a specific subtype of calcium channel, i.e., the N-type channel. In addition, using standard screening protocols, the libraries may be screened for compounds that block additional channels or receptors such as sodium channels, potassium channels and the like.

Methods of performing these screening functions are well known in the art. These methods can also be used for individually ascertaining the ability of a compound to agonize or antagonize the channel. Typically, the channel to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to antagonize the channel is measured in the presence of calcium, barium or other permeant divalent cation and the ability of the compound to interfere with the signal generated is measured using standard techniques. In more detail, one method involves the binding of radiolabeled agents that interact with the calcium channel and subsequent analysis of equilibrium binding measurements including, but not limited to, on rates, off rates, K_{d} values and competitive binding by other molecules.

Another method involves the screening for the effects of compounds by electrophysiological assay whereby individual cells are impaled with a microelectrode and currents through the calcium channel are recorded before and after application of the compound of interest.

Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel or as resting channel blockers. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz. Finally, resting channel blockers would diminish the peak current amplitude during the very first depolarization after drug application without additional inhibition during the depolarization.

Utility and Administration

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa., incorporated herein by reference.

In general, for use in treatment, the compounds of formula (1) may be used alone, as mixtures of two or more compounds of formula (1) or in combination with other pharmaceuticals. An example of other potential pharmaceuticals to combine with the compounds of formula (1) would include pharmaceuticals for the treatment of the same indication but having a different mechanism of action from N-type calcium channel blocking. For example, in the treatment of pain, a compound of formula (1) may be combined with another pain relief treatment such as an NSAID, or a compound which selectively inhibits COX-2, or an opioid, or an adjuvant analgesic such as an antidepressant. Another example of a potential pharmaceutical to combine with the compounds of formula (1) would include pharmaceuticals for the treatment of different yet associated or related symptoms or indications. Depending on the mode of administration, the compounds will be formulated into suitable compositions to permit facile delivery.

The compounds of the invention may be prepared and used as pharmaceutical compositions comprising an effective amount of at least one compound of formula (1) or formula (2) admixed with a pharmaceutically acceptable carrier or excipient, as is well known in the art. Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the dosage of the compounds of the invention is typically 0.01-15 mg/kg, preferably 0.1-10 mg/kg. However, dosage levels are highly dependent on the nature of the condition, drug efficacy, the condition of the patient, the judgment of the practitioner, and the frequency and mode of administration.

Synthesis of the Invention Compounds

The following examples are intended to illustrate the synthesis of a representative number of compounds. Accordingly, the following examples are intended to illustrate but not to limit the invention. Additional compounds not specifically exemplified may be synthesized using conventional methods in combination with the methods described hereinbelow.

### Example 1

### Synthesis of 2-(4-Benzhydryl-piperazin-1-yl)-N-(6-fluoro-benzothiazol-2-yl)-acetamide

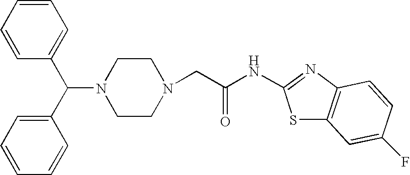

### A. Synthesis of ethyl-2-(4-benzhydrylpiperazin-1-yl)acetate

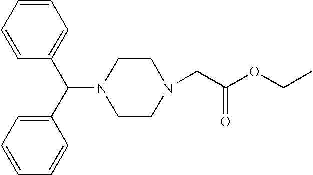

A mixture of diphenylmethylpiperazine (2.52 g, 10 mmol) in acetonitrile (20 ml), ethyl bromoacetate (1.67 g, 10 mmol), and anhydrous K₂CO₃ (1.38 g, 10 mmol) was refluxed under nitrogen for 3 hours. The mixture was then cooled and filtered and the solvent removed in vacuo. The residue was dissolved in ethyl acetate (50 ml) and washed with water (30 ml). Drying and removal of the solvent followed by chromatography (ethyl acetate: petroleum ether=1:3) afforded desired product (2.64 g) in 78% yield.

### B. Synthesis of 2-(4-benzhydrylpiperazin-1-yl)acetic acid

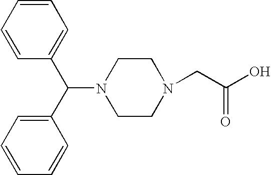

A mixture of ethyl 2-(4-benzhydrylpiperazin-1-yl)acetate 2.64 g (7.8 mmol), and LiOH.3H₂O (0.98 g, 23.4 mmol) in THF (45 ml), water (15 ml) and methanol (15 ml) was stirred at room temperature overnight. The mixture was then concentrated to remove the solvent. The residue was adjust to pH˜2 with 2N HCl and extracted with ethyl acetate (2×40 ml). The combined organic solution was dried with sodium sulfate and concentrated to give 2.2 g of desired product in yield 90%.

### C. Synthesis of 2-(4-Benzhydryl-piperazin-1-yl)-N-(6-fluoro-benzothiazol-2-yl)-acetamide

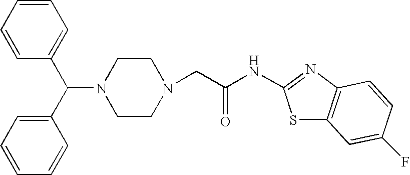

A solution of 2-(4-benzhydrylpiperazin-1-yl)acetic acid (155 mg, 0.5 mmol), 2-amino-6-fluorobenzothiazole (83 mg, 0.5 mmol), and EDC 191 mg (1 mmol) and DMAP (trace) in 5 ml dichloromethane was stirred at room temperature overnight. The reaction mixture was then concentrated, water was added and the reaction product was extracted with ethyl acetate (2×50 ml). The combined organic solution was dried over sodium sulfate and concentrated. The residue was then applied to flash column chromatography using ethyl acetate and petroleum ether (3:1) as eluents to give 100 mg of desired product in 45% yield.

### Example 2

Following the general procedures set forth in Example 1, the following compounds listed in Table 1 below were prepared. Mass spectrometry was employed with the final compound and at various stages throughout the synthesis as a confirmation of the identity of the product obtained (M+1). For the mass spectrometric analysis, samples were prepared at an approximate concentration of 1 μg/mL in acetonitrile with 0.1% formic acid. Samples were then manually infused into an Applied Biosystems API3000 triple quadrupole mass spectrometer and scanned in Q1 in the range of 50 to 700 m/z.

TABLE 1

| Cmpd | | | Mass Spec |
|---|---|---|---|
| No. | Name | Structure | (m/z) |
| 1 | 2-(4-benzhydrylpiperazin-1-yl)-N-(6- fluorobenzo[d]thiazol-2-yl)acetamide | 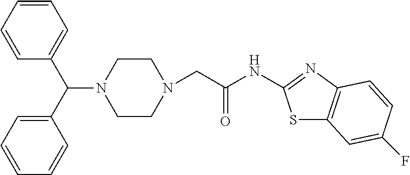 | 461.3 |
| 2 | 2-(4-(bis(4-fluorophenyl)methyl) piperazin-1-yl)-N-(6-fluorobenzo[d] thiazol-2-yl)acetamide | 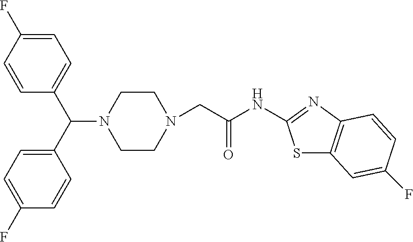 | 497 |
| 3 | 2-(4-benzhydrylpiperazin-1-yl)-N- (thiazol-2-yl)acetamide | 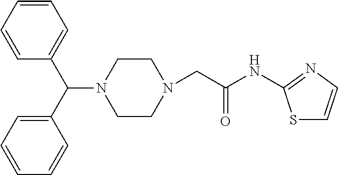 | 393.2 |
| 4 | 2-(4-(bis(4-fluorophenyl)methyl) piperazin-1-yl)-N-(thiazol-2- yl)acetamide | 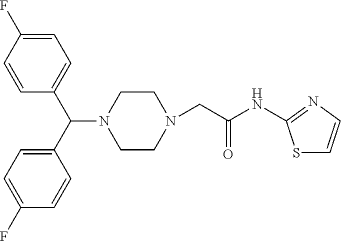 | 429.2 |
| 5 | 2-(4-benzhydrylpiperazin-1-yl)-N-(5- methylthiazol-2-yl)acetamide | 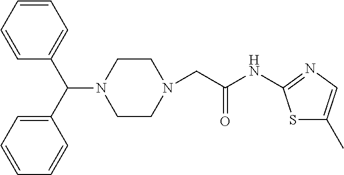 | 407.1 |
| 6 | 2-(4-(bis(4-fluorophenyl)methyl) piperazin-1-yl)-N-(5-methylthiazol-2- yl)acetamide | 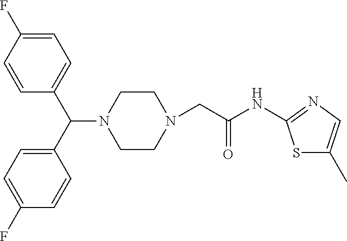 | 443.2 |
| 7 | 2-(4-benzhydrylpiperazin-1-yl)-N-(5- chlorothiazol-2-yl)acetamide | 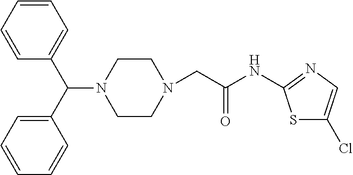 | 427.1 |
| 8 | 2-(4-(bis(4-fluorophenyl)methyl) piperazin-1-yl)-N-(5-chlorothiazol-2- yl)acetamide | 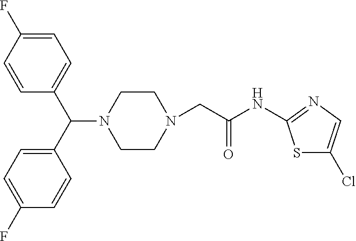 | 463.2 |
| 9 | 2-(4-benzhydrylpiperazin-1-yl)-N-(4-p- tolylthiazol-2-yl)acetamide | 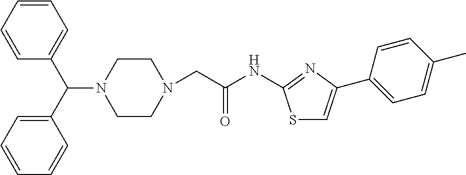 | 483.3 |
| 10 | 2-(4-(bis(4-fluorophenyl)methyl) piperazin-1-yl)-N-(4-p-tolylthiazol-2- yl)acetamide | 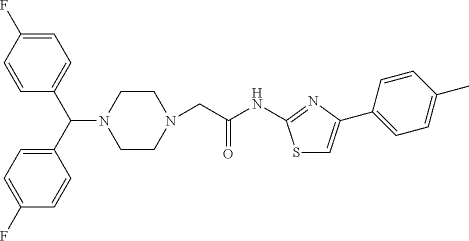 | 519.3 |
| 11 | 2-(4-benzhydrylpiperazin-1-yl)-N-(6- chlorobenzo[d]thiazol-2-yl)acetamide | 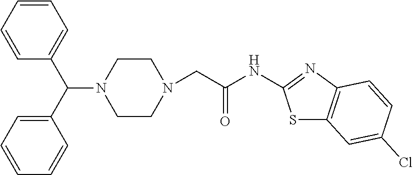 | 477.2 |
| 12 | 2-(4-(bis(4-fluorophenyl)methyl) piperazin-1-yl)-N-(6- chlorobenzo[d]thiazol-2-yl)acetamide | 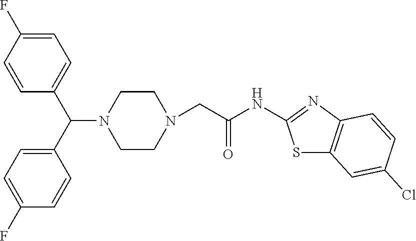 | 513.3 |
| 13 | 2-(4-benzhydrylpiperazin-1-yl)-N-(5- chlorobenzo[d]oxazol-2-yl)acetamide | 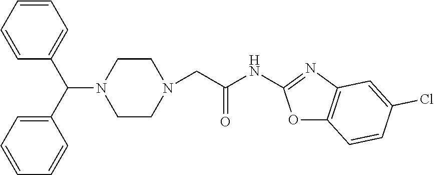 | 461.3 |
| 14 | 2-(4-benzhydrylpiperazin-1-yl)-N-(1H- benzo[d]imidazol-2-yl)acetamide | 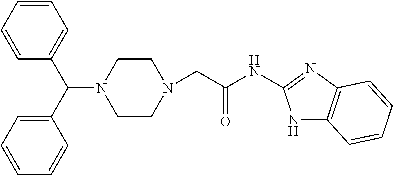 | 426.3 |
| 15 | N-(1H-benzo[d]imidazol-2-yl)-2-(4- (bis(4-fluorophenyl)methyl)piperazin-1- yl)acetamide | 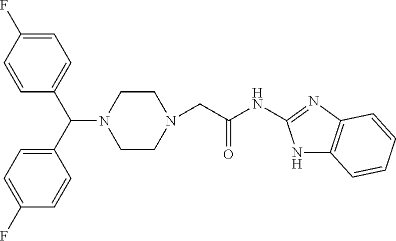 | 462.4 |
| 16 | 2-(4-benzhydrylpiperazin-1-yl)-N- (benzo[d]thiazol-2-yl)acetamide | 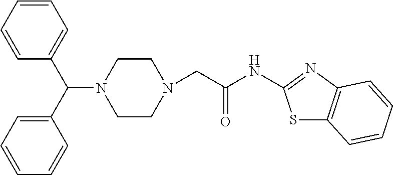 | 443.3 |
| 17 | N-(benzo[d]thiazol-2-yl)-2-(4-(bis(4- fluorophenyl)methyl)piperazin-1- yl)acetamide | 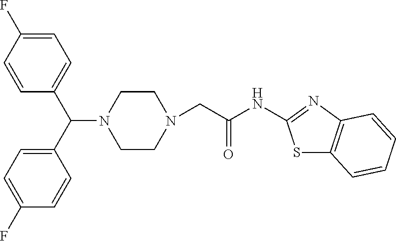 | 479.3 |
| 18 | 2-(4-benzhydrylpiperazin-1-yl)-N-(6- trifluoromethoxybenzo[d]thiazol-2- yl)acetamide | 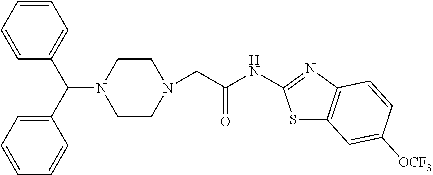 | 526.6 |
| 19 | 2-(4-benzhydrylpiperazin-1-yl)-N-(6- methoxybenzo[d]thiazol-2-yl)acetamide | 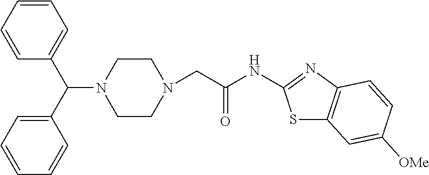 | 472.6 |
| 20 | 2-(4-benzhydrylpiperazin-1-yl)-N-(6- mesylbenzo[d]thiazol-2-yl)acetamide | 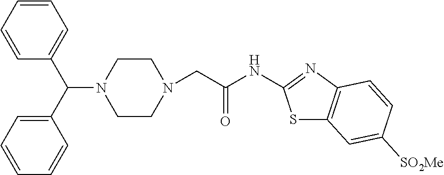 | 520.7 |

### Example 3

### N-type Channel Blocking Activities of Various Invention Compounds

A. Transformation of HEK Cells:

N-type calcium channel blocking activity was assayed in human embryonic kidney cells, HEK 293, stably transfected with the rat brain N-type calcium channel subunits (α_{1B}+α₂δ+β_{1b} cDNA subunits). Alternatively, N-type calcium channels (α_{1B}+α₂δ+β_{1b} cDNA subunits), L-type channels (α_{1C}+α₂δ+β_{1b} cDNA subunits) and P/Q-type channels (α_{1A}+α₂δ+β_{1b} cDNA subunits) were transiently expressed in HEK 293 cells. Briefly, cells were cultured in Dulbecco's modified eagle medium (DMEM) supplemented with 10% fetal bovine serum, 200 U/ml penicillin and 0.2 mg/ml streptomycin at 37° C. with 5% CO₂. At 85% confluency cells were split with 0.25% trypsin/1 mM EDTA and plated at 10% confluency on glass coverslips. At 12 hours the medium was replaced and the cells transiently transfected using a standard calcium phosphate protocol and the appropriate calcium channel cDNA's. Fresh DMEM was supplied and the cells transferred to 28° C./5% CO₂. Cells were incubated for 1 to 2 days prior to whole cell recording.

B. Measurement of Inhibition

Whole cell patch clamp experiments were performed using an Axopatch 200B amplifier (Axon Instruments, Burlingame, Calif.) linked to a personal computer equipped with pCLAMP software. The external and internal recording solutions contained, respectively, 5 mM BaCl₂, 10 mM MgCl₂, 10 mM HEPES, 40 mM TEACl, 10 mM glucose, 87.5 mM CsCl (pH 7.2) and 108 mM CsMS, 4 mM MgCl₂, 9 mM EGTA, 9 mM HEPES (pH 7.2). Currents were typically elicited from a holding potential of −80 mV to +10 mV using Clampex software (Axon Instruments). Typically, currents were first elicited with low frequency stimulation (0.067 Hz) and allowed to stabilize prior to application of the compounds. The compounds were then applied during the low frequency pulse trains for two to three minutes to assess tonic block, and subsequently the pulse frequency was increased to 0.2 Hz to assess frequency dependent block. Data were analyzed using Clampfit (Axon Instruments) and SigmaPlot 4.0 (Jandel Scientific).

Specific data obtained for N-type channels are shown in Table 2 below.

TABLE 2
N-type Calcium Channel Block

| Compound | IC₅₀ @ −80 mV (μM) | IC₅₀ @ −50 mV (μM) |
|---|---|---|
| 1 | 0.77 | 0.40 |
| 2 | 0.92 | 0.49 |
| 4 | 3.40 | 1.20 |
| 5 | 1.95 | 0.78 |
| 8 | 1.04 | 0.53 |
| 9 | 6.40 | 2.50 |
| 10 | 3.54 | 1.91 |
| 11 | 3.40 | 1.60 |
| 12 | 4.58 | 2.56 |
| 14 | 0.29 | 0.25 |
| 16 | 1.21 | 0.60 |
| 17 | 2.53 | 0.53 |
| 18 | 2.90 | 1.16 |
| 19 | 3.16 | 1.54 |
| 20 | 3.13 | 1.64 |

### Example 4

### Activity of Invention Compounds in Formalin-Induced Pain Model

The effects of intrathecally delivered compounds of the invention on the rat formalin model can also be measured. The compounds can be reconstituted to stock solutions of approximately 10 mg/ml in propylene glycol. Typically eight Holtzman male rats of 275-375 g size are randomly selected per test article.

The following study groups can be used, with test article, vehicle control (propylene glycol) and saline delivered intraperitoneally (IP):

TABLE 3
Formalin Model Dose Groups

| Test/Control Article | Dose | Route | Rats per group |
|---|---|---|---|
| Compound | 30 mg/kg | IP | 6 |
| Propylene glycol | N/A | IP | 4 |
| Saline | N/A | IP | 7 |

N/A = Not Applicable

Prior to initiation of drug delivery baseline behavioral and testing data can be taken. At selected times after infusion of the Test or Control Article these data can then be again collected.

On the morning of testing, a small metal band (0.5 g) is loosely placed around the right hind paw. The rat is placed in a cylindrical Plexiglas chamber for adaptation a minimum of 30 minutes. Test Article or Vehicle Control Article is administered 10 minutes prior to formalin injection (50 μl of 5% formalin) into the dorsal surface of the right hindpaw of the rat. The animal is then placed into the chamber of the automated formalin apparatus where movement of the formalin injected paw is monitored and the number of paw flinches tallied by minute over the next 60 minutes (Malmberg, A. B., et al., Anesthesiology (1993) 79:270-281).

Results can be presented as Maximum Possible Effect±SEM, where saline control=100%.

### Example 5

### Spinal Nerve Ligation Model of Neuropathic Pain

Spinal nerve ligation (SNL) injury can be induced using the procedure of Kim and Chung, (Kim, S. H., et al., Pain (1992) 50:355-363) in male Sprague-Dawley rats (Harlan; Indianapolis, Ind.) weighing 200 to 300 grams. Anesthesia is induced with 2% halothane in O₂ at 2 Umin and maintained with 0.5% halothane in O₂. After surgical preparation of the rats and exposure of the dorsal vertebral column from L₄ to S₂, the L₅ and L₆ spinal nerves are tightly ligated distal to the dorsal root ganglion using 4-0 silk suture. The incision is closed, and the animals are allowed to recover for 5 days. Rats that exhibit motor deficiency (such as paw-dragging) or failure to exhibit subsequent tactile allodynia are excluded from further testing. Sham control rats undergo the same operation and handling as the experimental animals, but without SNL.

The assessment of tactile allodynia consists of measuring the withdrawal threshold of the paw ipsilateral to the site of nerve injury in response to probing with a series of calibrated von Frey filaments. Each filament is applied perpendicularly to the plantar surface of the ligated paw of rats kept in suspended wire-mesh cages. Measurements are taken before and after administration of drug or vehicle. Withdrawal threshold is determined by sequentially increasing and decreasing the stimulus strength (“up and down” method), analyzed using a Dixon non-parametric test (Chaplan S. R., et al., J Pharmacol Exp Ther (1994) 269:1117-1123), and expressed as the mean withdrawal threshold.

The method of Hargreaves and colleagues (Hargreaves, K., et al., Pain (1988) 32:77-8) can be employed to assess paw-withdrawal latency to a thermal nociceptive stimulus. Rats are allowed to acclimate within a plexiglas enclosure on a clear glass plate maintained at 30° C. A radiant heat source (i.e., high intensity projector lamp) is then activated with a timer and focused onto the plantar surface of the affected paw of nerve-injured or carrageenan-injected rats. Paw-withdrawal latency can be determined by a photocell that halted both lamp and timer when the paw is withdrawn. The latency to withdrawal of the paw from the radiant heat source is determined prior to carrageenan or L5/L5 SNL, 3 hours after carrageenan or 7 days after L5/L6 SNL but before drug and after drug administration. A maximal cut-off of 40 seconds is employed to prevent tissue damage. Paw withdrawal latencies can be thus determined to the nearest 0.1 second. Reversal of thermal hyperalgesia is indicated by a return of the paw withdrawal latencies to the pre-treatment baseline latencies (i.e., 21 seconds). Anti nociception is indicated by a significant (p<0.05) increase in paw withdrawal latency above this baseline. Data is converted to % anti hyperalgesia or % anti nociception by the formula: (100×(test latency−baseline latency)/(cut-off−baseline latency) where cut-off is 21 seconds for determining anti hyperalgesia and 40 seconds for determining anti nociception.

What is claimed:

1. A method to treat acute pain or chronic pain comprising administering to a subject in need thereof an effective amount of a compound, wherein said compound is of the formula: 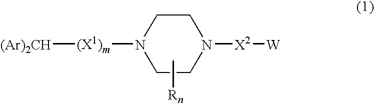
or a pharmaceutically acceptable salt or a conjugate thereof
W is an optionally substituted imidazolyl, thiazolyl, oxazolyl, benzimidazolyl, benzothiazolyl or benzoxazolyl;
X¹ is an optionally substituted alkylene (1-6C), alkenylene (2-6C), alkynylene (2-6C), heteroalkylene (2-6C), heteroalkenylene (2-6C), or heteroalkynylene (2-6C);
X² is an optionally substituted alkylene (3-6C) or heteroalkylene (2-6C) that is attached to the carbon atom of W between the heteroatoms in the 5-membered ring of W;
each Ar is independently an optionally substituted aromatic or heteroaromatic ring;
each R is independently ═O, ═NOR′, halo, CN, OR′, SR′, SOR′, SO₂R′, NR′₂, NR′(CO)R′, or NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-10C); or R may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-1° C.), heteroaryl (5-12C), O-aryl (6-1° C.), O-heteroaryl (5-12C) and C6-C12-aryl-C1-C6-alkyl;
n is 0-4;
m is 0-1; and
wherein the optional substituents on each Ar and W are independently selected from halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-1° C.); or the optional substituent may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12C), O-aryl (6-10C), O-heteroaryl (5-12C) and C₆-C₁₂-aryl-C₁-C₆-alkyl; and
wherein the optional substituents on each X¹ and X² are independently selected from ═O, ═NOR′, halo, CN, OR′, SR′, SOR′, SO₂R′, NR′₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-10C); or R may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12C), O-aryl (6-1° C.), O-heteroaryl (5-12C) and C6-C12-aryl-C1-C6-alkyl;
wherein each optional substituent on an aromatic or heteroaromatic group is independently selected from halo, CN, NO₂, CF₃, COOR′, CONR′₂, OR′, SR′, SOR′, SO₂R′, NR′₂, NR′(CO)R′, or NR′SO₂R′, wherein each R′ is independently H or alkyl (1-6C), heteroaryl (5-12C), or aryl (6-10C); or the substituent may be selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12C), O-aryl (6-10C), O-heteroaryl (5-12C) and C6-C12-aryl-C1-C6-alkyl; and
each optional substituent on an alkyl, alkenyl, alkynyl, heteroalkyl, heteroalkenyl or heteroalkynyl group is independently selected from ═O, ═NOR′, halo, CN, OR′, SR′, SOR′, SO₂R′, NR′₂, NR′(CO)R′, or NR′SO₂R′, wherein each R′ is independently H or is selected from alkyl (1-6C), heteroaryl (5-12C), and aryl (6-10C); or it may be alkyl (1-6C), alkenyl (2-6C), or alkynyl (2-6C), heteroalkyl (2-6C), heteroalkenyl (2-6C), heteroalkynyl (2-6C), aryl (6-10C), heteroaryl (5-12C), O-aryl (5-10C), O-heteroaryl (5-12C) and C6-C12-aryl-C1-C6-alkyl.

2. The method of claim 1 wherein said condition is modulated by N-type calcium channel activity.

3. The method of claim 1 wherein said condition is acute pain.

4. The method of claim 1, wherein said condition is chronic pain.

5. The method of claim 1 wherein m is 0.

6. The method of claim 1 or claim 5 wherein n is 0 or 1 or 2.

7. The method of claim 1, wherein Ar and W are unsubstituted.

8. The method of claim 1, wherein at least one Ar or W is substituted by at least one of halo, alkyl(1-4C), or an optionally substituted aryl (6-10C).

9. The method of claim 8 wherein at least one Ar or W is substituted by at least one of chloro, fluoro, methyl or tolyl.

10. The method of claim 1, wherein each Ar is independently an optionally substituted phenyl.

11. The method of claim 1, wherein X² is an optionally substituted heteroalkylene (2-6C).

12. The method of claim 1 wherein X² is an optionally substituted heteroalkylene (2-4C).

13. The method of claim 1 or wherein X² is CH₂CONH.

14. The method of claim 1 wherein the compound is:
2-(4-benzhydrylpiperazin-1-yl)-N-(6-fluorobenzo[d]thiazol-2-yl)acetamide
2-(4-(bis(4-fluorophenyl)methyl)piperazin-1-yl)-N-(6-fluorobenzo[d]thiazol-2-yl)acetamide
2-(4-benzhydrylpiperazin-1-yl)-N-(thiazol-2-yl)acetamide
2-(4-(bis(4-fluorophenyl)methyl)piperazin-1-yl)-N-(thiazol-2-yl)acetamide
2-(4-benzhydrylpiperazin-1-yl)-N-(5-methylthiazol-2-yl)acetamide
2-(4-(bis(4-fluorophenyl)methyl)piperazin-1-yl)-N-(5-methylthiazol-2-yl)acetamide
2-(4-benzhydrylpiperazin-1-yl)-N-(5-chlorothiazol-2-yl)acetamide
2-(4-(bis(4-fluorophenyl)methyl)piperazin-1-yl)-N-(5-chlorothiazol-2-yl)acetamide
2-(4-benzhydrylpiperazin-1-yl)-N-(4-p-tolylthiazol-2-yl)acetamide
2-(4-(bis(4-fluorophenyl)methyl)piperazin-1-yl)-N-(4-p-tolylthiazol-2-yl)acetamide
2-(4-benzhydrylpiperazin-1-yl)-N-(6-chlorobenzo[d]thiazol-2-yl)acetamide
2-(4-(bis(4-fluorophenyl)methyl)piperazin-1-yl)-N-(6-chlorobenzo[d]thiazol-2-yl)acetamide
2-(4-benzhydrylpiperazin-1-yl)-N-(5-chlorobenzo[d]oxazol-2-yl)acetamide
2-(4-benzhydrylpiperazin-1-yl)-N-(1H-benzo[d]imidazol-2-yl)acetamide
N-(1H-benzo[d]imidazol-2-yl)-2-(4-(bis(4-fluorophenyl)methyl)piperazin-1-yl)acetamide
2-(4-benzhydrylpiperazin-1-yl)-N-(benzo[d]thiazol-2-yl)acetamide
N-(benzo[d]thiazol-2-yl)-2-(4-(bis(4-fluorophenyl)methyl)piperazin-1-yl)acetamide;
2-(4-benzhydrylpiperazin-1-yl)-N-(6-trifluoromethoxybenzo[d]thiazol-2-yl)acetamide;
2-(4-benzhydrylpiperazin-1-yl)-N-(6-methoxybenzo[d]thiazol-2-yl)acetamide;
2-(4-benzhydrylpiperazin-1-yl)-N-(6-mesylbenzo[d]thiazol-2-yl)acetamide;
or a pharmaceutically acceptable salt of one of these.

15. A compound of the formula: 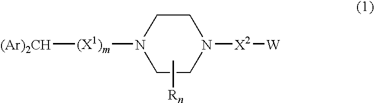
or a pharmaceutically acceptable salt or a conjugates thereof
W is an optionally substituted imidazolyl, oxazolyl, benzimidazolyl, or benzoxazolyl;
X¹ is an optionally substituted alkylene (1-6C), alkenylene (2-6C), alkynylene (2-6C), heteroalkylene (2-6C), heteroalkenylene (2-6C), or heteroalkynylene (2-6C);
X² is an optionally substituted alkylene (3-6C) or heteroalkylene (2-6C) that is attached to the carbon atom of W between the heteroatoms in the 5-membered ring of W;
each Ar is independently an optionally substituted aromatic or heteroaromatic ring;
each R is independently ═O, ═NOR′, halo, CN, OR′, SR′, SOR′, SO₂R′, NR′₂, NR′(CO)R′, or NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-1° C.); or R may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-1° C.), heteroaryl (5-12C), O-aryl (6-10C), O-heteroaryl (5-12C) and C6-C12-aryl-C1-C6-alkyl;
n is 0-4;
m is 0-1; and
wherein the optional substituents on each Ar and W are independently selected from halo, CN, NO₂, CF₃, OCF₃, COOR′, CONR′₂, OR′, SR′, SOR′, SO₂R′, NR₁₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-10C); or the optional substituent may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12C), O-aryl (6-10C), O-heteroaryl (5-12C) and C6-C12-aryl-C1-C6-alkyl; and
wherein the optional substituents on each X¹ and X² are independently selected from ═O, ═NOR′, halo, CN, OR′, SR′, SOR′, SO₂R′, NR′₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), heteroalkynyl (2-6C), heteroaryl (5-12C), and aryl (6-10C); or R may be an optionally substituted group selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12C), O-aryl (6-1° C.), O-heteroaryl (5-12C) and C6-C12-aryl-C1-C6-alkyl;
wherein each optional substituent on an aromatic or heteroaromatic group is independently selected from halo, CN, NO₂, CF₃, COOR′, CONR′₂, OR′, SR′, SOR′, SO₂R′, NR′₂, NR′(CO)R′, or NR′SO₂R′, wherein each R′ is independently H or alkyl (1-6C), heteroaryl (5-12C), or aryl (6-10C); or the substituent may be selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12C), O-aryl (6-10C), O-heteroaryl (5-12C) and C6-C12-aryl-C1-C6-alkyl; and
each optional substituent on an alkyl, alkenyl, alkynyl, heteroalkyl, heteroalkenyl or heteroalkynyl group is independently selected from ═O, ═NOR′, halo, CN, OR′, SR′, SOR′, SO₂R′, NR′₂, NR′(CO)R′, or NR′SO₂R′, wherein each R′ is independently H or is selected from alkyl (1-6C), heteroaryl (5-12C), and aryl (6-10C); or it may be alkyl (1-6C), alkenyl (2-6C), or alkynyl (2-6C), heteroalkyl (2-6C), heteroalkenyl (2-6C), heteroalkynyl (2-6C), aryl (6-10C), heteroaryl (5-12C), O-aryl (5-10C), O-heteroaryl (5-12C) and C6-C12-aryl-C1-C6-alkyl.

16. The compound of claim 15 wherein m is 0.

17. The compound of claim 15, wherein n is 0 or 1 or 2.

18. The compound of claim 15, wherein Ar and W are unsubstituted.

19. The compound of any of claim 15, wherein each Ar is independently an optionally substituted phenyl.

20. The compound of claim 15, wherein at least one of Ar and W is substituted by at least one of halo, alkyl(1-4C), or an optionally substituted aryl (6-10C).

21. The compound of claim 20 wherein at least one of Ar and W is substituted by at least one of chloro, fluoro, methyl or tolyl.

22. The compound of claim 15, wherein X² is an optionally substituted heteroalkylene (2-6C).

23. The compound of claim 22 wherein X² is an optionally substituted heteroalkylene (2-4C).

24. The compound of claim 15, wherein X² is CH₂CONH.

25. The compound of claim 15 wherein the compound is:
2-(4-benzhydrylpiperazin-1-yl)-N-(1H-benzo[d]imidazol-2-yl)acetamide
N-(1H-benzo[d]imidazol-2-yl)-2-(4-(bis(4-fluorophenyl)methyl)piperazin-1-yl)acetamide
or a pharmaceutically acceptable salt of one of these.

26. A pharmaceutical composition which comprises the compound of claim 15 in admixture with a pharmaceutically acceptable excipient.
